(19) United States
(12) Patent Application Publication
(10) Pub. No.: US 2022/0390333 A1
(43) Pub. Date: Dec. 8, 2022

(54) ADIPOSE TISSUE PARTICLE PROCESSING, TRANSFER AND STORAGE SYSTEM

(71) Applicant: Roger S. Hogue, Maple Grove, MN (US)

(72) Inventor: Roger S. Hogue, Maple Grove, MN (US)

(21) Appl. No.: 17/807,621

(22) Filed: Jun. 17, 2022

Related U.S. Application Data
(63) Continuation-in-part of application No. 17/184,399, filed on Feb. 24, 2021.
Application No. 17/184,399 is a continuation-in-part of application No. 17/119,930, filed on Dec. 11, 2020.
(60) Provisional application No. 62/946,701, filed on Dec. 11, 2019.

(51) Int. Cl. G01N 1/14; G01N 1/40

(52) U.S. Cl. CPC G01N 1/14; G01N 1/4077; G01N 2001/4088; G01N 2001/1418

(57) ABSTRACT

A method and system for processing an adipose tissue material sample to create and collect fat aspirate particles having particle diameters less than or equal to a selected size are disclosed. A filter screen assembly having an interface part, a closed end opposite to the interface part, and a screen portion therebetween is inserted through a first port in a lid of a container, with the interface part providing access to an interior of the container for insertion of a transfer cannula connected to a syringe filled with the adipose tissue material. The screen portion includes a plurality of apertures having diameters of the selected size. Adipose tissue material is expelled from the transfer cannula through the apertures of the filter screen assembly into the container to create and collect the fat aspirate particles having particle diameters less than or equal to the selected size.

### CROSS-REFERENCE TO RELATED APPLICATION(S)

This application is a continuation-in-part of U.S. application Ser. No. 17/184,399 entitled “ADIPOSE TISSUE PARTICLE PROCESSING, TRANSFER AND STORAGE SYSTEM” filed on Feb. 24, 2021 by R. Hogue, which is a continuation-in-part of U.S. application Ser. No. 17/119,930 entitled “ADIPOSE TISSUE PARTICLE PROCESSING, TRANSFER AND STORAGE SYSTEM” filed on Dec. 11, 2020 by R. Hogue, which in turn claims priority to U.S. Provisional Application No. 62/946,701 entitled “ADIPOSE TISSUE PARTICLE PROCESSING, TRANSFER AND STORAGE SYSTEM” filed on Dec. 11, 2019 by R. Hogue. U.S. application Ser. No. 17/184,399, U.S. application Ser. No. 17/119,930 and U.S. Provisional Application No. 62/946,701 are all incorporated by reference herein in their entireties.

### BACKGROUND

The present invention relates to systems and methods for processing, transferring and storing adipose tissue, such as fat aspirate obtained by liposuction.

Adipose tissue, or body fat, is loose connective tissue composed mostly of adipocytes, such as fat cells, along with a vast array of regenerative cell populations, including adipose-derived stem cells or mesenchymal stem cells, which have tremendous potential benefits for human tissue regeneration.

In order to harvest adipose tissue or fat aspirate containing regenerative call populations such as adipocyte-derived stem cells, a minimally-invasive treatment that uses tumescent liposuction techniques to harvest fat tissue as lipoaspirate can be used. Additional processing steps are routinely used following the initial harvesting procedure (i.e., tumescent liposuction), including fat aspirate particle sizing (micro-fragmenting or micronizing), filtering (removal of sinuate, connective tissue strands, and coarse debris), separating and concentrating (via gravity decanting or centrifugation to separate, isolate and remove water, blood, and oil from viable fat aspirate particles) in order to create an autologous fat graft that can be used for injection or deployment during an autologous fat grafting (fat transfer) treatment for the purpose of aesthetic (cosmetic) and/or regenerative purposes. Autologous fat grafting and/or autologous regenerative treatments containing autologous fat aspirate particles are used for cosmetic and/or therapeutic rejuvenation, restoration, and repair of aging or degenerative tissues such as the skin, hair, face, body, breasts, cleavage, dorsum of hands, soft tissue, wounds, scars, musculoskeletal tissues, vocal cords, and genitalia.

Currently, several procedures exist for processing (sizing, filtering, separating, and concentrating) fat aspirate particles. One such procedure involves placing the fat aspirate inside a chamber having many small steel balls immersed in saline. The chamber is then shaken whereby the steel balls micro-fragment the fat aspirate while the saline cleans it. This procedure can result in pulverization and indiscriminate sizing of the fat particles due to the high variability in shaking the chamber. Other procedures entail passing the fat aspirate back-and-forth many times across a mesh-like surface or screen with a square-shaped pattern to micronize the particles by using luer-to-luer syringe transfer. This processing can severely mechanically traumatize the fat aspirate particles and destroy the adipocytes, as well as be time consuming and physically straining. As a result, there is a need for systems and methods that result in improved processing (sizing, filtering, separating and concentrating) of fat aspirate obtained by liposuction harvesting using precision outer dimensional sizing and filtering to create optimally purified and viable micro-fragmented adipose tissue for clinical deployment in fat transfer cosmetic and/or regenerative procedures.

### BRIEF DESCRIPTION OF THE DRAWINGS

The drawings are intended to illustrate embodiments of, but not to limit, the present invention. In the drawings, like reference characters denote corresponding features consistently throughout similar embodiments.

FIGS. 1A and 1B illustrate an adipose tissue particle processing system according to an embodiment of the present invention.

FIGS. 2A and 2B illustrate the assembly of the adipose tissue particle processing system shown in FIGS. 1A and 1B.

FIG. 3 illustrates a filter screen assembly for use in an adipose tissue particle processing system according to an embodiment of the present invention.

FIGS. 4A and 4B illustrate a cap/bushing for use in an adipose tissue particle processing system according to an embodiment of the present invention.

FIG. 5 illustrates a transfer cannula that may be used with an adipose tissue particle processing system according to an embodiment of the present invention.

FIG. 6 illustrates a cannula cleaner that may be used with an adipose tissue particle processing system according to an embodiment of the present invention.

FIG. 7 is a flow diagram illustrating a process for sizing adipose tissue particles through a filter screen assembly with a transfer cannula according to an embodiment of the present invention.

FIGS. 8A-8E illustrate a transfer cannula at various depths in a filter screen according to an embodiment of the present invention.

FIG. 9 is a flow diagram illustrating a process of successively reducing the size of adipose tissue particles by repeatedly performing the process of FIG. 7.

FIG. 10 illustrates an adipose tissue particle processing system according to an alternative configuration, with a syringe and transfer cannula inserted into a filter screen assembly that is supported inside a container.

FIG. 11 illustrates the adipose tissue particle processing system shown in FIG. 10 with the syringe and transfer cannula removed.

FIG. 12 illustrates an exemplary configuration of the filter screen assembly in more detail

### DETAILED DESCRIPTION

The present disclosure provides an adipose tissue particle processing system that allows a physician to process (precision size by micro-fragmentation, and filter and remove debris and strands) adipose tissue into controlled fat aspirate particle sizes for use in autologous fat transfer and/or autologous regenerative treatments containing the autologous fat aspirate particles.

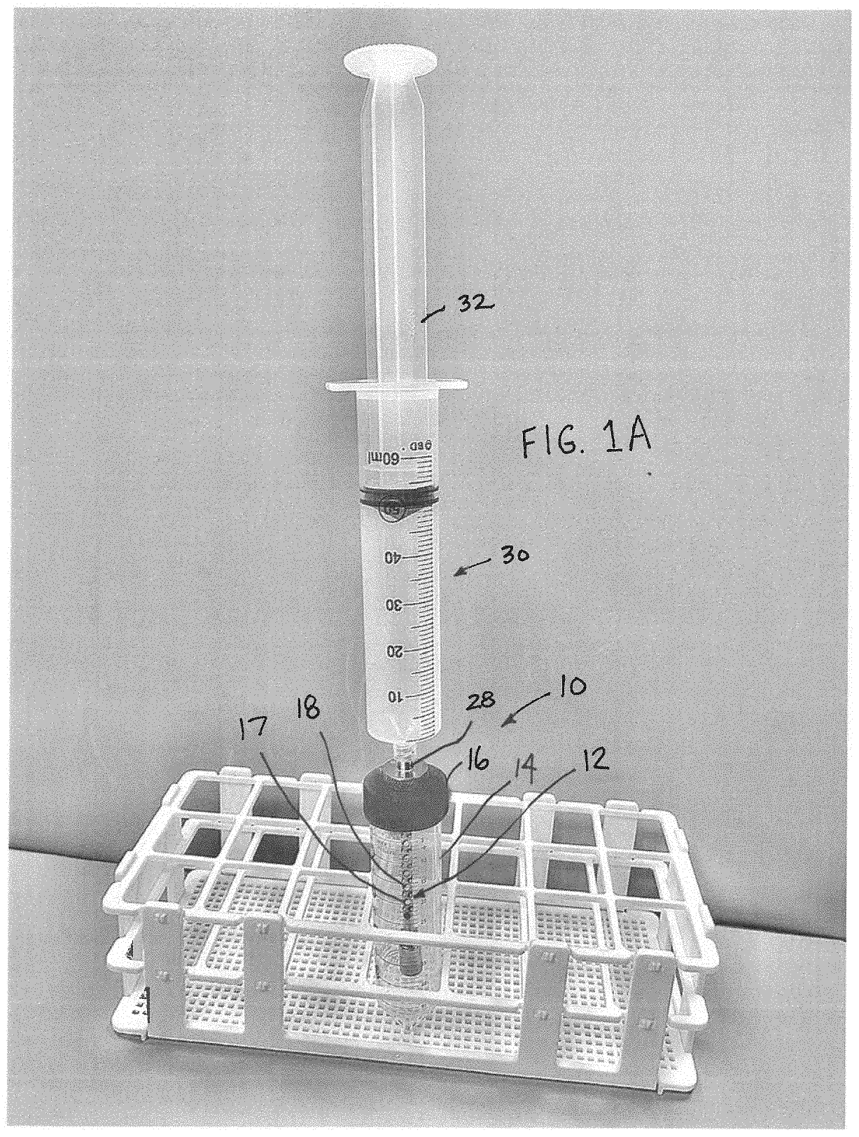

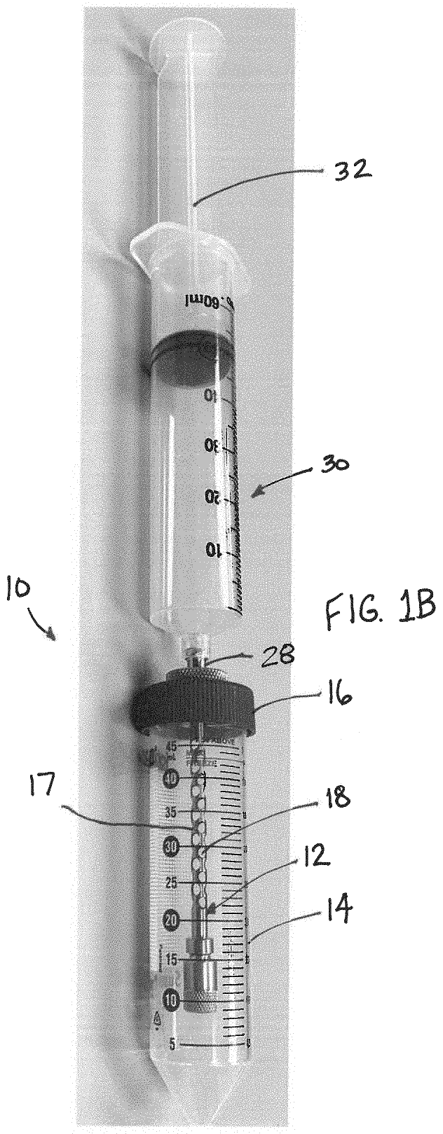

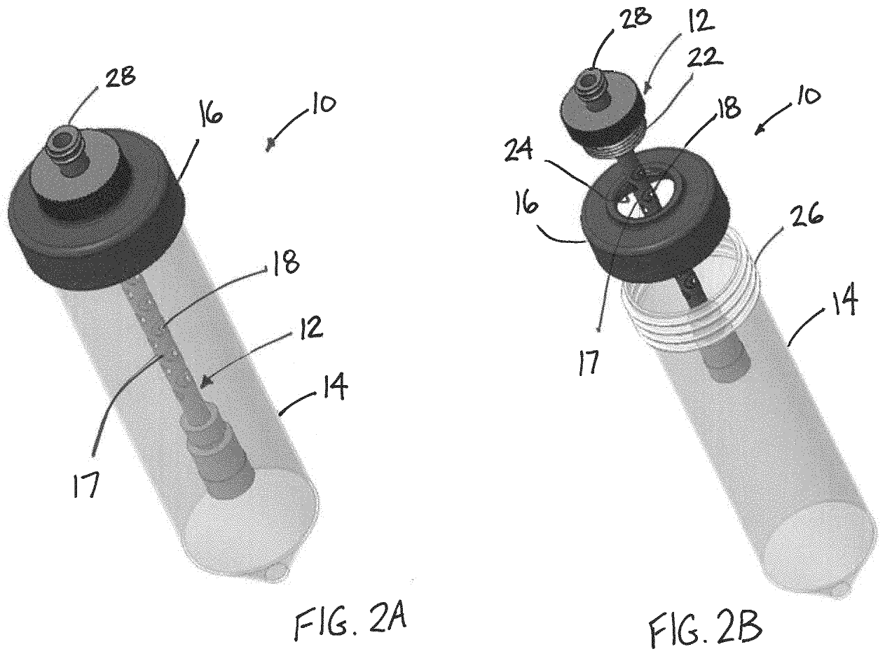

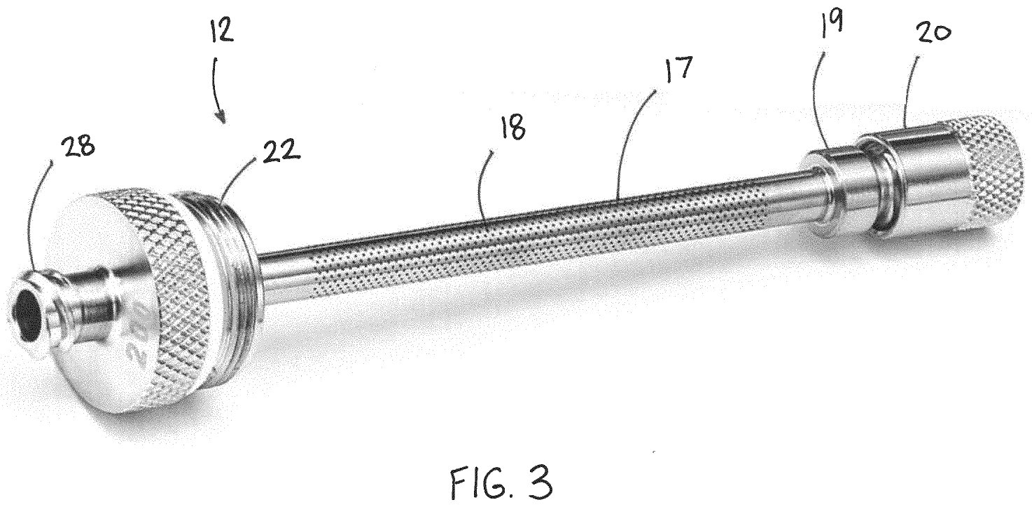

FIGS. 1A and 1B illustrate adipose tissue particle processing system 10 that includes a filter screen assembly 12 (which is also shown in more detail in FIG. 3) positioned to extend into a container (for example, plastic centrifuge tube 14, although other suitable containers may be used in other embodiments) through cap/bushing 16. FIGS. 2A and 2B illustrate the assembly of the filter screen assembly 12 extending through cap/bushing 16 into the interior of centrifuge tube 14 of adipose tissue particle processing system 10. Filter screen assembly 12 includes screen portion 17 that is made up of a plurality of apertures 18 that have diameters selected for processing adipose tissue into controlled fat aspirate particle sizes. In the embodiment shown (see FIG. 3 in particular), the distal end of filter screen assembly 12 has female luer fitting 19, which allows male luer cap 20 to be attached to close the distal end of filter screen assembly 12 during use. Other methods or configurations for providing a closed distal end of filter screen assembly 12 during use may be used in alternative embodiments.

Also, in the embodiment shown (see FIGS. 2A and 2B in particular), filter screen assembly 12 includes male threads 22 near its proximal end, and cap/bushing 16 includes female threads 24 that are configured to receive male threads 22 of filter screen assembly 12, to secure filter screen assembly 12 to cap/bushing 16 so that screen portion 17 is suspended in the interior of centrifuge tube 14 when cap/bushing 16 is positioned on the top of centrifuge tube 14. In other embodiments, filter screen assembly 12 could alternatively be connected to a luer fitting or threaded fitting on cap/bushing 16, or could be integrally formed (e.g., by welding or adhesive connection) with cap/bushing 16.

In the embodiment shown, centrifuge tube 14 is made of clear plastic, and has a tapered configuration from its top (where cap/bushing 16 is provided) to its bottom (where a conical tapered end is provided). This is a common configuration for a plastic centrifuge tube, which is readily manufactured by injection molding, for example. In an alternative embodiment, a zero-draft, cylindrical plastic centrifuge tube may be constructed and used, which has no taper from the top to the bottom of the tube, and which has a flat bottom surface rather than a conical tapered end. With such a construction, the cylindrical plastic centrifuge tube could be used with the system described in U.S. patent application Ser. No. 16/295,695 entitled “Aspirating Separated Liquid Components From A Vessel” filed on Mar. 7, 2019, which is incorporated by reference herein in its entirety. In the system described in U.S. patent application Ser. No. 16/295,695, a diaphragm is slidably coupleable to the hollow inner portion of the centrifuge tube, and allows liquid contained in the centrifuge tube to be selectively and controllably aspirated out of the centrifuge tube through the diaphragm.

Centrifuge tube 14 shown in FIGS. 1A-2B is a 50 mL tube, but it should be understood that larger or smaller sizes and volumes of containers./centrifuge tubes may be used in other embodiments.

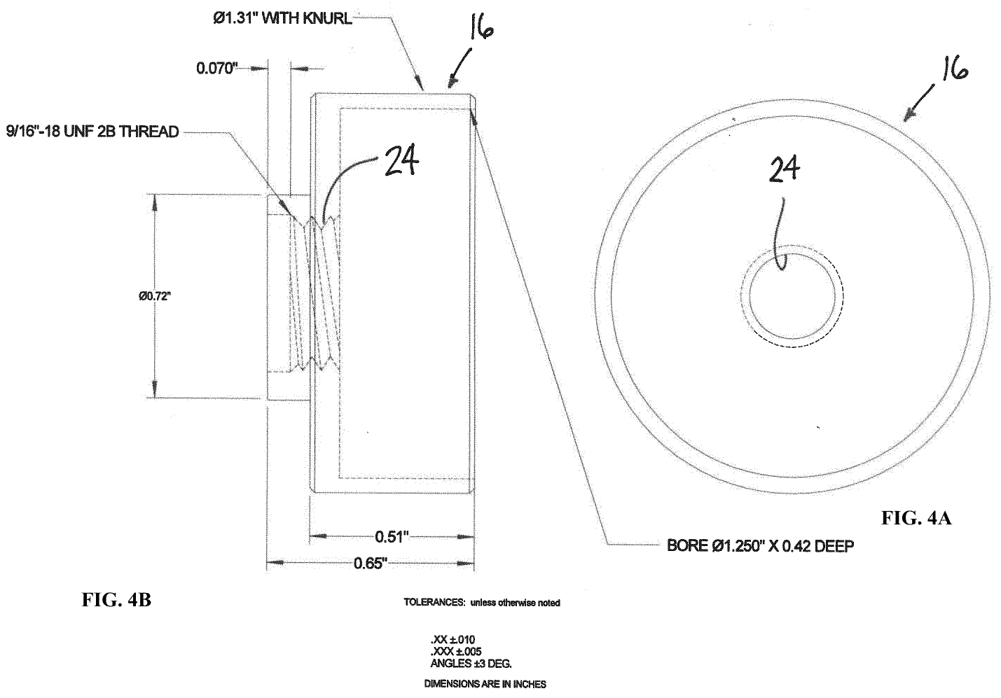

In the embodiment shown (see FIGS. 4A and 4B in particular), cap/bushing 16 is made of plastic, and has a threaded central aperture (having female threads 24) that engages with male threads 22 of filter screen assembly 12, so that screen portion 17 of filter screen assembly 12 is supported and suspended inside centrifuge tube 14. In the embodiment shown, cap/bushing 16 is formed with a configuration that allows cap/bushing 16 to slip over male threads 26 at the top of centrifuge tube 14 (rather than threadedly engaging with male threads 26 at the top of centrifuge tube 14, as a standard lid for centrifuge tube 14 would do). With cap/bushing 16 configured to slip over male threads 26 at the top of centrifuge tube 14, venting is provided to allow depositing and aspirating of material to/from centrifuge tube 14, due to the non-airtight fitting between cap/bushing 16 and centrifuge tube 14. In alternative embodiments, cap/bushing 16 may have female threads which are threadedly engaged with male threads 26 at the top of centrifuge tube 14, thereby providing an airtight coupling between them, and cap/bushing 16 may be further designed to include venting apertures in its disc-shaped face, with a suitable air-permeable membrane, such as a 0.2-micron filter in some examples, to prevent liquid material from escaping through cap/bushing 16. In some alternative embodiments, cap/bushing 16 may be formed of stainless steel (with any of the variations of configurations described above), and may be a reusable component.

Exemplary dimensions for the various features of cap/bushing 16 are shown in FIGS. 4A and 4B. It should be understood that these dimensions are provided to illustrate one example of cap/bushing 16, and that the configuration of the features of cap/bushing 16 may have other dimensions either larger or smaller than the dimensions listed in other embodiments.

Apertures 18 in screen portion 17 of filter screen assembly 12 may be formed in by laser drilling in some embodiments. Example sizes/diameters of apertures 18 may be as large as 4.0 millimeters, as small as 0.2 millimeters, any size/diameter in between, or sizes/diameters larger than 4.0 millimeters or smaller than 0.2 millimeters, depending on the application in which the adipose tissue particle processing system 10 is used.

In one example, screen portion 17 of filter screen assembly 12 may have an outer diameter of about 0.259 inches (about 6.58 millimeters). In other examples, screen portion 17 of filter screen assembly 12 may have larger or smaller radial dimensions. In some embodiments, filter screen assembly 12 is composed of stainless steel.

In various embodiments, some of the components of adipose tissue particle sizing system 10 are designed to be reusable components (typically made of stainless steel), while other components are designed to be single-use, disposable components (typically made of plastic). In this context, components described as reusable are capable of being cleaned and sterilized multiple times, such as be a sterilizing autoclave, by enzyme treatment, or by other methods, while single-use, disposable components are provided in sterile packaging for a single use.

In operation, as shown in FIGS. 1A and 1B, female luer fitting 28 at the proximal end of filter screen assembly 12 is configured to allow coupling to the outlet of syringe 30, which can contain tissue material to be processed by adipose tissue particle processing system 10. Once syringe 30 is coupled to adipose tissue particle processing system 10, tissue material may be transferred into adipose tissue particle processing system 10 by pressing plunger 32 of syringe 30. This causes adipose tissue material to pass into the interior of screen portion 17 of filter screen assembly 12, with the distal end of filter screen assembly 12 being closed by luer cap 20, so that fat aspirate particles in the adipose tissue material are forced to pass from the interior of filter screen assembly 12 through apertures 18 of screen portion 17 into the interior of centrifuge tube 14. The fat aspirate particles are effectively “filtered” and “sized” (micro-fragmented) by sieve filtering and shearing force by apertures 18 of screen portion 17 of filter screen assembly 12, to a size that is determined by the size of apertures 18, while undesired sinuate, connective tissue strands, and coarse debris are not able to pass through apertures 18.

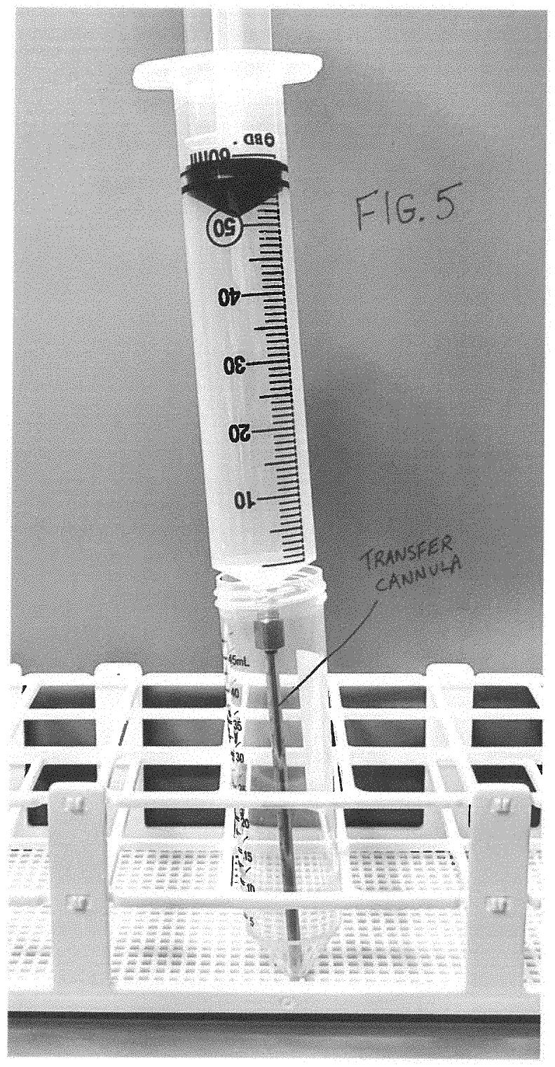

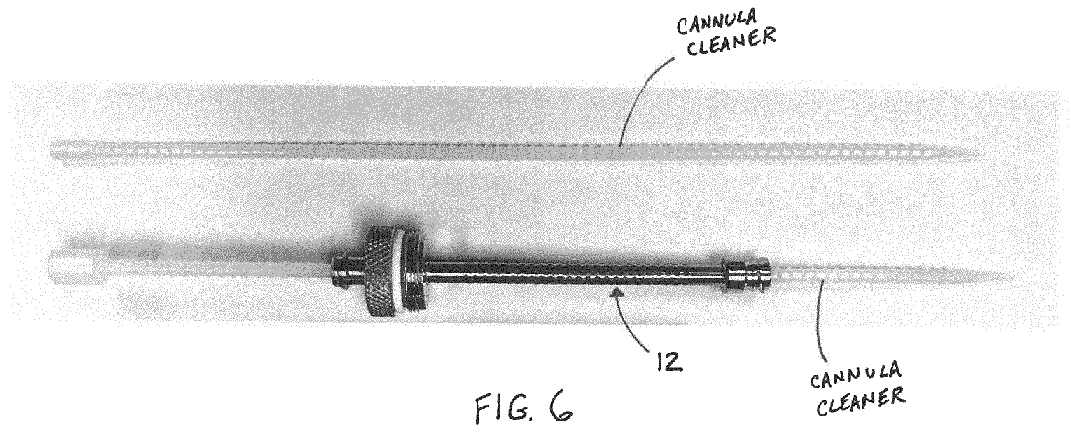

Once the micro-fragmented “sized” fat aspirate particles are transferred through screen portion 17 of filter screen assembly 12 into centrifuge tube 14, then centrifuge tube 14 may be prepared for centrifugation, by removing components of adipose tissue particle processing system 10, and replacing cap/bushing 16 with a conventional threaded lid. After the micro-fragmented fat aspirate particles are separated by either gravity decantation, or by centrifugation in a centrifuge system, various separated components may be aspirated from centrifuge tube 14. In some embodiments, aspiration may be performed by inserting a transfer cannula into the interior of centrifuge tube 14 and aspirating material through the transfer cannula with a syringe coupled to the transfer cannula (as illustrated in FIG. 5 as transfer cannula 60). The transfer cannula shown in FIG. 5 may be a 6-inch or 12-inch length cannula with a female luer-lock connector on its proximal end and an approximately 0.146-inch (3.7 mm) outer diameter cylindrical tubular blunt tip on its distal end In other embodiments, where centrifuge tube 14 has a zero-draft, cylindrical configuration, the method described in U.S. patent application Ser. No. 16/295,695 may be used, where a diaphragm is slidably coupleable to the hollow inner portion of centrifuge tube 14, and allows liquid contained in centrifuge tube 14 to be selectively and controllably aspirated out of centrifuge tube 14 through the diaphragm.

Filter screen assembly 12 may be cleaned after use by removing male luer cap 20 from the distal end, and inserting a cannula cleaner that is configured with projecting surfaces such as convex fins into the interior of filter screen assembly 12. Cleaning is performed by scraping, dislodging, and removing debris and contaminants when making direct physical contact with the interior of a cannula device when moved back-and-forth following use of the cannula device, to be moved back and forth to cause frictional engagement with filter screen assembly 12 for cleaning. The cannula cleaner may be made of medical-grade nylon in some embodiments. In some embodiments, the cannula cleaner may be configured as shown and described in U.S. Provisional Application No. 62/855,167 entitled “Method and Apparatus for Cleaning the Interior Cannula of Suction Lipoplasty Cannula Devices and Adipose Tissue and/or Fluid Particle Sizing Devices,” filed on May 31, 2019, which is hereby incorporated by reference.

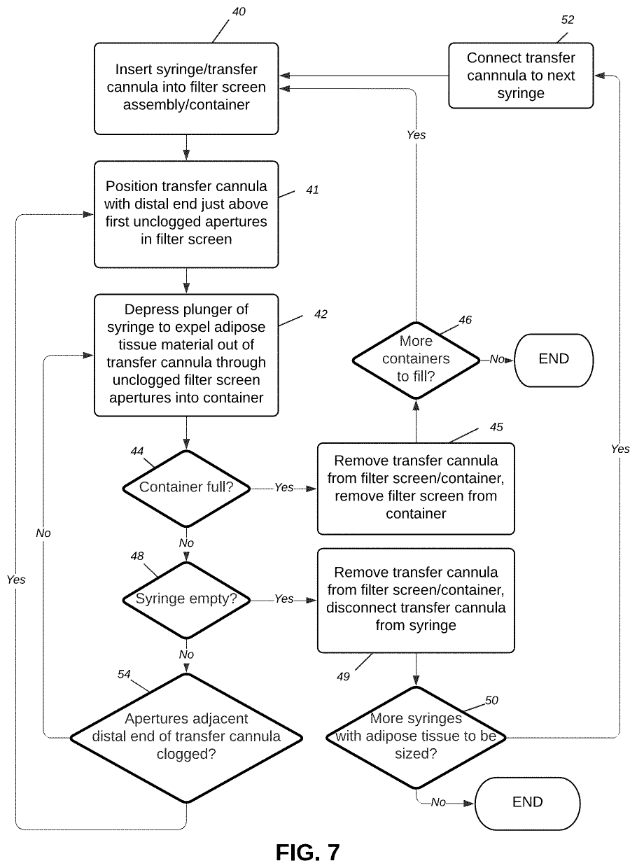

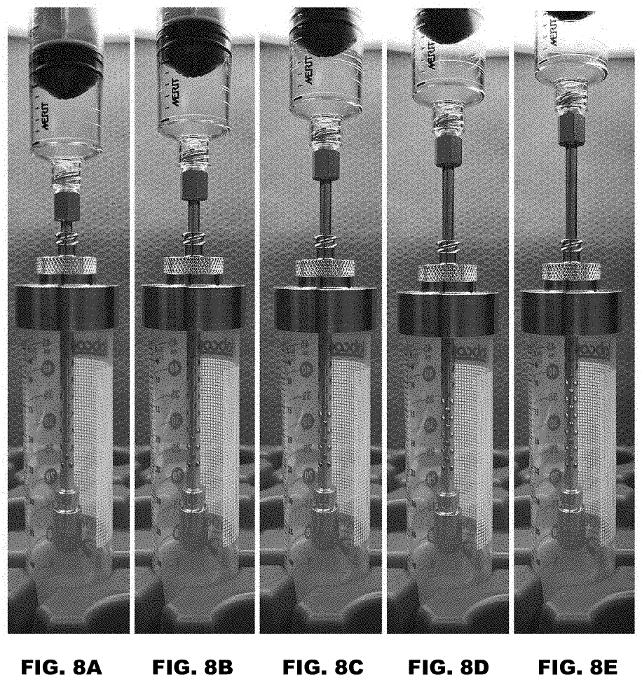

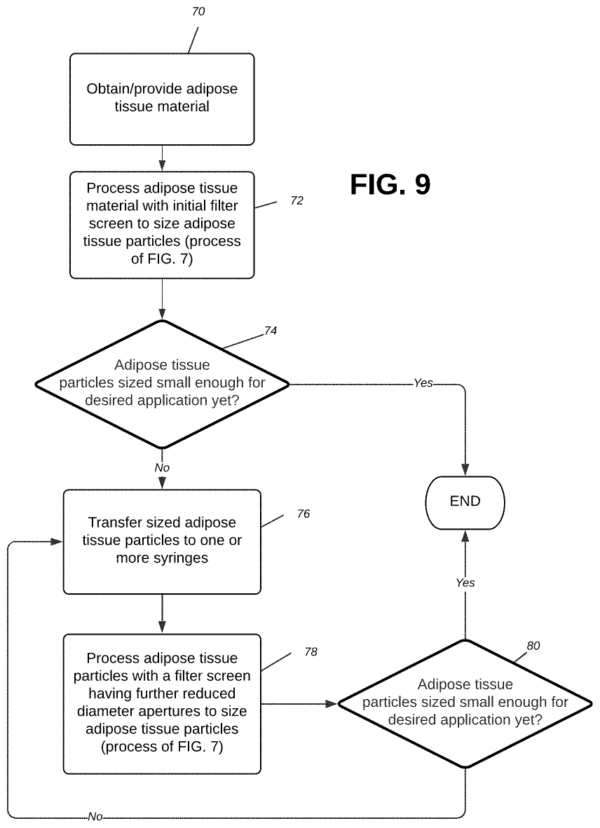

FIGS. 7 and 8A-8E illustrate an exemplary process for transferring adipose tissue particles through a filter screen assembly, in which partial clogging of the filter screen assembly is dealt with in a manner that still allows the adipose tissue particles to pass through the filter screen assembly. FIG. 7 is a flow diagram illustrating the process for transferring adipose tissue particles through filter screen assembly 12 using a successively withdrawn transfer cannula 60. FIGS. 8A-8E illustrate transfer cannula 60 at various depths in filter screen assembly 12, to show how transfer cannula 60 is successively withdrawn during the course of the process to transfer adipose tissue through filter screen assembly 12 into container 14. While container 14 is described and illustrated in the present disclosure as being a plastic centrifuge tube, it should be understood that container 14 may be any suitable container for collecting processed fat aspirate particles that pass through the filter screen assembly 12. Container 14 should be a transparent container, to allow a clinician to see the interior of container 14 during processing and control the process accordingly.

As an initial step, a collected sample of adipose tissue particles is provided in one or more syringes, and a transfer cannula is attached to the output of a syringe, so that the adipose tissue sample in the syringe can be output through the transfer cannula. FIGS. 8A-8E show syringe 30 connected to transfer cannula 60. In various embodiments, transfer cannula may be the same transfer cannula shown and described above with respect to FIG. 5, or may be a different transfer cannula that has the same or a different length. Then, to begin the process of transferring adipose tissue particles into container 14, transfer cannula 60 is inserted through the opening of female luer fitting 28 at the proximal end of filter screen assembly 12, and is extended into the interior of filter screen assembly 12 (step 40, FIG. 7). This is shown in FIG. 8A with transfer cannula 60 extending into the interior of filter screen assembly 12 until reaching or nearly reaching luer cap 20 at the distal end of filter screen assembly 12, where transfer cannula 60 can be seen through all of apertures 18 in filter screen assembly 12. In some embodiments, transfer cannula 60 has a length that is sufficiently long to reach all the way to luer cap 20 at the distal end of filter screen assembly, while in some other embodiments, transfer cannula 60 has a length that is specially configured so that the distal end of transfer cannula 60, when fully inserted into filter screen assembly 12, does not reach luer cap 20 at the distal end of filter screen assembly. In some exemplary embodiments, there is a clearance of a few microns (approximately 5 microns in one particular example, to provide a friction fit) between the outer diameter of transfer cannula 60 and the inner diameter of the opening of female luer fitting 28, to allow for easy but secure insertion. In some exemplary embodiments, there is a clearance of a few microns (approximately 5 microns in one particular example, to provide a friction fit) between the outer diameter of transfer cannula 60 and the inner diameter of filter screen assembly 12. This ensures that adipose tissue particles expelled from the distal end of transfer cannula 60 cannot migrate up inside filter screen assembly 12 beyond the distal end of transfer cannula 60, and will instead be forced to pass through apertures 18 of filter screen assembly 12 that are below the distal end of transfer cannula 60.

In order for adipose tissue particles to be able to pass through the apertures of filter screen assembly 12, transfer cannula 60 is positioned by a clinician so that the distal end or transfer cannula is located just above the first unclogged apertures 18 of filter screen assembly 12 (step 41, FIG. 7). This is done by a clinician holding the syringe 30 with the transfer cannula 60 attached thereto, and is shown in FIG. 8B, where transfer cannula 60 can be seen through all but the bottom (distal) 20% (approximately) of apertures 18 in filter screen assembly 12. As was noted above, in some embodiments, the initial position of the distal end of transfer cannula 60 may be similar to the illustration in FIG. 8B, with some of the bottom apertures 18 in filter screen assembly 12 unobstructed by transfer cannula 60, due to the specially configured length of transfer cannula 60. While in the position shown in FIG. 8B, a clinician then depresses the plunger of syringe 30 to expel the adipose tissue particles out of transfer cannula 60, and into the interior of screen portion 17 of filter screen assembly 12, with the distal end of filter screen assembly 12 being closed by luer cap 20. This causes fat aspirate particles in the adipose tissue material that exit from transfer cannula 60 to be forced to pass from the interior of filter screen assembly 12 through apertures 18 of screen portion 17 into the interior of container 14 (step 42, FIG. 7). Specifically, the fat aspirate particles in the adipose tissue material that exit from transfer cannula 60 pass through the bottom (distal) 20% of apertures 18 that are not obstructed by transfer cannula 60. The fat aspirate particles are effectively “filtered” and “sized” (micro-fragmented) by sieve filtering and shearing force by apertures 18 of screen portion 17 of filter screen assembly 12, to a size that is determined by the size of apertures 18, while undesired sinuate, connective tissue strands, and coarse debris are not able to pass through apertures 18.

While adipose tissue particles are being transferred into container 14 through filter screen assembly 12, the clinician monitors the process to determine whether the container 14 is full (step 44). When container 14 is full, the particle transfer process is paused. The clinician then removes transfer cannula 60 from filter screen assembly 12 and removes filter screen assembly 12 from container 14 (step 45), and determines whether there are additional containers that need to be filled (step 46). If no further containers need to be filled, the process is over. If there are additional containers to be filled, filter screen assembly 12 is inserted into the next container 14 (step 47), and the process returns to step 40, in which the clinician inserts transfer cannula 60 attached to syringe 30 into the interior of filter screen assembly 12.

Also, while adipose tissue particles are being transferred into container 14 through filter screen assembly 12, the clinician monitors the process to determine whether syringe 30 is empty (step 48). If syringe 30 is empty, the clinician removes transfer cannula 60 from filter screen assembly 12 and disconnects transfer cannula 60 from (empty) syringe 30 (step 49). The clinician then determines whether there are additional syringes containing adipose tissue that needs to be sized (step 50). If there are no further adipose tissue samples that need to be sized, the process is over. If there are additional syringes containing adipose tissue to be sized, the clinician attaches transfer cannula 60 to the next syringe 30 (step 52), and the process returns to step 40, in which the clinician inserts transfer cannula 60 attached to syringe 30 into the interior of filter screen assembly 12.

If syringe 30 is not empty, the clinician continues to monitor the process to determine whether apertures 18 of filter screen assembly 12 adjacent to the distal end of transfer cannula 60 are clogged (step 54). Depending on the nature of the adipose tissue being processed, the volume of syringe 30, the volume of container 14, and the total amount of samples of adipose tissue particles to be sized, apertures 18 in filter screen assembly 12 may begin to become clogged with sinuate or other material that is filtered by filter screen assembly 12 during the adipose tissue sizing process. If the monitoring clinician determines that apertures 18 are not clogged, then the clinician continues to depress the plunger of syringe 30 to expel adipose tissue through filter screen assembly 12, illustrated by the process looping back to step 42 and continuing to be monitored by decision steps 44, 48 and 54. If apertures 18 are clogged, the clinician repositions transfer cannula 60 within filter screen assembly 12 so that the distal end of transfer cannula 60 is located just above higher, unlogged apertures 18 of filter screen assembly (step 41). This concept is illustrated in FIGS. 8B-8E, where transfer cannula 60 is successively withdrawn to positions where approximately 20% (FIG. 8B), 40% (FIG. 8C), 60% (FIG. 8D) and 80% (FIG. 8E) of apertures 80 are unobstructed by transfer cannula 60. As filter screen assembly 12 becomes more clogged during the process of transferring adipose tissue material to container 14, the clinician will insert transfer cannula 60 to a lesser and lesser extent within filter screen assembly 12 (as shown by the illustrations from left to right in FIGS. 8A-8E), to ensure that adipose tissue particles are able to pass through unclogged apertures 18 and be sized by filter screen assembly 12.

If all of the apertures 18 of filter screen assembly 12 are clogged (that is, the top/proximal-most apertures 18 are clogged), then it is necessary to replace filter screen assembly 12 with a new filter screen assembly 12 in order to continue the process. That is, the process is stopped in order to insert a new filter screen assembly 12 into container 14, and the process then begins again by inserting transfer cannula 60 through the opening of female luer fitting 28 at the proximal end of filter screen assembly 12, to extend into the interior of filter screen assembly 12 (step 40, FIG. 7).

The process shown in FIG. 7 may be (and typically must be) repeated for multiple successive sizing steps, to reduce the size of adipose tissue particles from a relatively large size to a significantly smaller size, while filtering larger particles, sinuate, etc., and while avoiding damage to the fat aspirate particles that are sized. This successive/repeated sizing process is illustrated in FIG. 9. As shown in FIG. 9, adipose tissue material is initially obtained/provided (step 70), and then the process of FIG. 7 is performed using an initial filter screen (having apertures of an initial size) to size adipose tissue particles to maximum diameters that correspond to the size of the apertures (step 72). It is then determined whether the processed adipose tissue particles are sized small enough for the desired application (step 74). If the adipose tissue particles have a sufficiently small size, the process ends, and the adipose tissue particles may be used for the desired application. If the adipose tissue particles not yet small enough for the desired application, then the adipose tissue particles are collected and transferred into one or more syringes (step 76), and are then processed again by the process of FIG. 7, using a filter screen having apertures of a size that is smaller than the apertures of the filter screen used in the previous processing step (step 78). Then, it is determined whether the processed adipose tissue particles are sized small enough for the desired application (step 80). If the adipose tissue particles have a sufficiently small size, the process ends, and the adipose tissue particles may be used for the desired application. If the adipose tissue particles not yet small enough for the desired application, then the process returns to step 76, to repeat the steps of transferring the adipose tissue particles to one or more syringes and processing the adipose tissue particles using a filter screen having successively smaller apertures, until the adipose tissue particles have a size that is sufficiently small for the desired application.

Several examples of the successive/repeated process illustrated in FIG. 9 may be considered. In a first example, an initial adipose tissue sample may first be sized via the process of FIG. 7 (step 72) using a filter screen assembly having apertures with a diameter of 2000 microns. Then, the “2000-micron-sized” particles may be further sized via the process of FIG. 7 using a filter screen assembly having apertures with a diameter of 1000 microns (step 78), the “1000-micron-sized” particles may be further sized via the process of FIG. 7 using a filter screen assembly having apertures with a diameter of 500 microns (step 78), the “500-micron-sized” particles may be further sized via the process of FIG. 7 using a filter screen assembly having apertures with a diameter of 300 microns (step 78), and the “300-micron-sized” particles may be further sized via the process of FIG. 7 using a filter screen assembly having apertures with a diameter of 200 microns (step 78). The resulting “200-micron-sized” particles may be used for any of a number of appropriate medical procedures in which fat aspirate particles of that size are desirable and appropriate. In a second example, the sizes of the apertures of filter screens that are successively used to reduce the size of the adipose tissue particles may be 2500 microns-1200 microns-700 microns-500 microns-300 microns-200 microns. In a third example, the sizes of the apertures of filter screens that are successively used to reduce the size of the adipose tissue particles may be 3000 microns-1500 microns-800 microns-500 microns-300 microns-200 microns. It should be understood that the examples of sizes of particles and diameters of apertures 18 of filter screen assembly 12 are provided for illustration purposes, and any suitable sizes of particles can be obtained, using any suitable aperture diameters of filter screen assembly 12, and any number of successive sizing operations, in various embodiments.

In order to transfer intermediate “sized” adipose tissue particles from a centrifuge tube into a syringe for further sizing, in some embodiments, a clinician may insert a clean filter screen assembly 12, having apertures 18 with the same diameter as was just used to size the adipose tissue particles, into container 14, with luer cap 20 removed (leaving an open distal end). Then, the clinician takes an empty syringe 30 having transfer cannula 60 attached, and inserts transfer cannula 60 through filter screen assembly 12 to a position at or near the bottom of container 14. In other embodiments, the clinician may simply insert transfer cannula 60 attached to an empty syringe 30 through the opening of cap/bushing 16 into the interior of container 14, without using filter screen assembly 12. In either case, next, the clinician pulls up the plunger of syringe 30, while slightly repositioning transfer cannula 60 within container 14 as needed, to draw the adipose tissue particles up into syringe 30. This allows the clinician to fill one or more syringes with the intermediate “sized” adipose tissue particles for further sizing (particle size reduction) in subsequent processes as shown in FIG. 7 and FIG. 9.

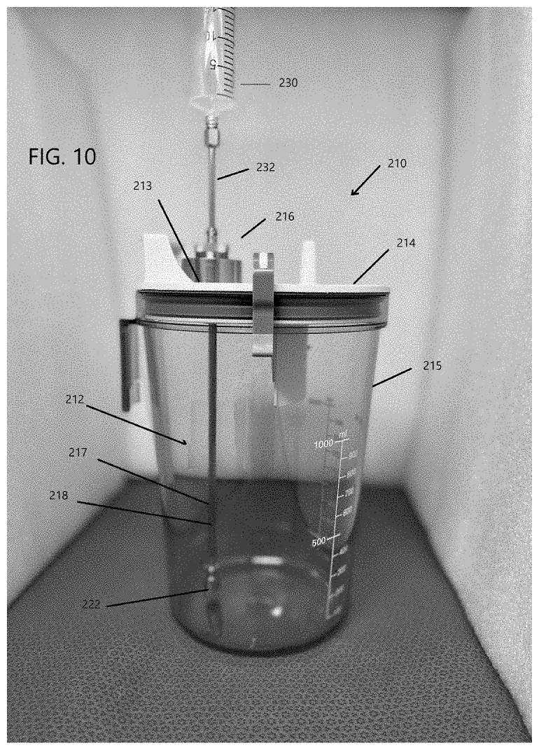

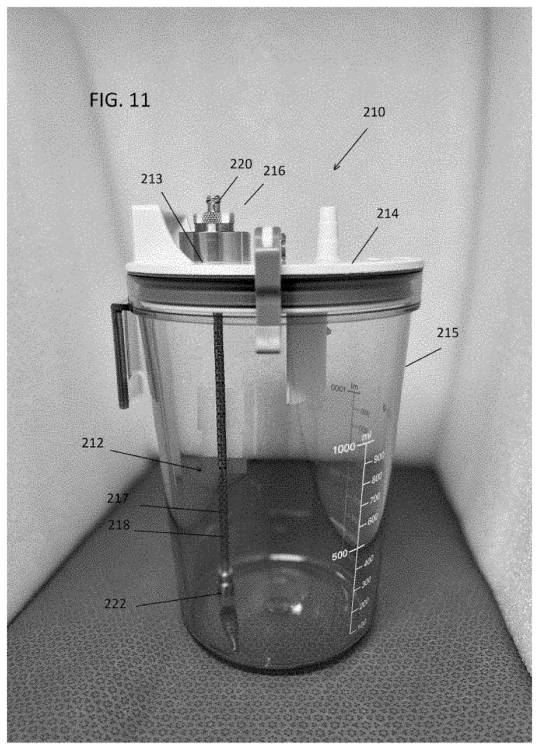

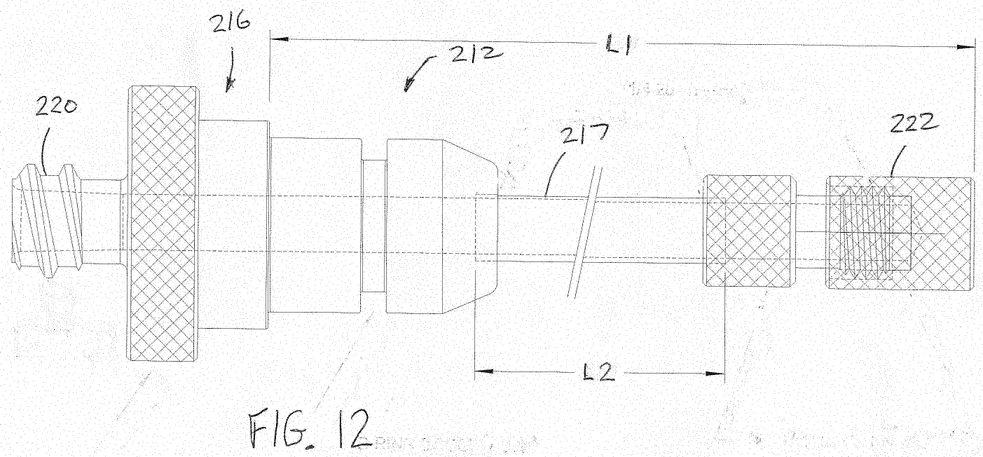

FIGS. 10 and 11 illustrate an adipose tissue particle processing system 210 according to an alternative configuration (FIG. 10 shows adipose tissue particle processing system 210 with a syringe and transfer cannula connected, and FIG. 11 shows adipose tissue particle processing system 210 with the syringe and transfer cannula disconnected/removed). Adipose tissue particle processing system 210 includes filter screen assembly 212 that is insertable through a port 213 in lid 214 of canister 215. In the embodiment shown in FIG. 12, filter screen assembly 212 includes mating plug 216 connected to a top end of screen portion 217 of filter screen assembly 212 (as viewed in FIGS. 10 and 11), and mating plug 216 is friction fit with the port 213 in lid 214. This configuration allows syringe 230, via transfer cannula 232, to be coupled through a central opening of mating plug 216 into the interior of screen portion 217 of filter screen assembly 212. Mating plug 216 serves as an interface part between screen portion 217 and transfer cannula 232 attached to syringe 230. Screen portion 217 of filter screen assembly 212 is positioned inside canister 215, and has its bottom end (as viewed in FIGS. 10 and 11) closed, such as by threaded cap 222 in one exemplary embodiment.

FIG. 12 illustrates an exemplary configuration of filter screen assembly 212 in more detail. In the embodiment shown in FIG. 12, the central opening of mating plug 216 is configured as a female luer opening 220, which has a diameter sufficient to receive a transfer cannula without requiring a luer lock coupling. Screen portion 217 of filter screen assembly 212 is attached to mating plug 216 (or a supporting structure coupled thereto), for being suspended into the interior of canister 215 during use. At the end of screen portion 217 opposite to mating plug 216, a threaded portion is provided for securing of threaded cap 222 to close the lumen formed by screen portion 217 of filter screen assembly 212. This will force adipose tissue that is injected into the interior of screen portion 217 to be forced through apertures 218 for collection in canister 215.

In operation, syringe 230 (which may be a 30 ml syringe in one exemplary embodiment) is filled with adipose tissue to be filtered/sized, such as by drawing adipose tissue through a suction port from a separate container (not shown) through transfer cannula 232. The adipose tissue to be filtered/sized may consist of particles that have a larger size than would be desirable for autologous fat transfer, so that filtering/sizing is needed to reduce the particle size of the adipose tissue and remove sinuate from the tissue—in one example embodiment, the initial size of the particles of the adipose tissue to be filtered/sized that is contained in syringe 230 is about 2-3 mm. Once syringe 230 is filled with adipose tissue to be filtered/sized, transfer cannula 232 may be inserted through the central opening of mating plug 216 into the interior of screen portion 217 of filter screen assembly 212. With the transfer cannula 232 held in position inside filter screen assembly 212, the plunger of syringe 230 may be depressed, to push the adipose tissue to be filtered/sized out of transfer cannula 232, where the adipose tissue will then pass through the apertures 218 of screen portion 217 of filter screen assembly 212 and be filtered and resized to a smaller particle size, into the interior of canister 215. This process can be repeated multiple times through the same filter screen assembly 212, with the transfer cannula 232 successively positioned adjacent to apertures 218 of screen portion 217 that remain unclogged, in the general manner shown in FIG. 7 and explained in the description of FIG. 7 above, until all or nearly all of the apertures 218 are clogged with sinuate debris. Then, filter screen assembly 212 may be removed from port 213 in lid 214, and a replacement filter screen assembly 212 may be inserted into port 213 in its place, for further filtering/sizing of adipose tissue and collection into canister 215. The entire process may be repeated until a desired amount of filtered/sized adipose tissue is obtained in canister 215.

This filtering/sizing operation is similar to that described above with respect to adipose tissue processing system 10, and allows a large volume of adipose tissue to be filtered/sized and collected in canister 215, using multiple filter screen assemblies 212 as each individual filter screen assembly becomes clogged with sinuate debris. For example, canister 215 may have a volume of 1000 mL in some embodiments. This may be particularly advantageous for larger volume autologous fat transfer applications, such as breast or buttock augmentation. For example, the filtering/sizing operation may involve adipose tissue filled in syringe 230 with a particle size of about 2-3 mm, and apertures 218 in screen portion 217 of filter screen assembly 212 may have a diameter of 1500 microns, so that adipose tissue particles are precision sized to 1500 microns with sinuate connective material removed, suitable for use in a breast or buttock augmentation procedure. In other examples, different sized apertures may be used, such as 1200 microns or other sizes. It is also possible to repeat the entire process to further reduce the size of the adipose tissue by successively using a filter screen assembly 212 with smaller apertures, if desired, as described above in relation to FIG. 9.

Canister 215 may include a port near its bottom for removal of fat from canister 215 after adipose tissue has been filtered/sized. Also, port 213 may be equipped with or receive a seal in order to seal lid 214 of canister 215 after filter screen assembly 212 is removed. Once the desired amount of filtered/sized adipose tissue is obtained inside canister 215, a tube may be coupled to the port near the bottom of canister 215 (preferably with port 213 sealed) in order to decant the liquid (typically a small amount of excess water that was harvested as part of the adipose tissue filtering/sizing operation) contained in canister 215.

In some embodiments, filter screen assembly 212 shown in FIGS. 10-12 may be configured to be longer than filter screen assembly 10 described previously, in part because of the larger interior volume (and height) of canister 215. For example, filter screen assembly may extend a total length from mating plug 216 to threaded cap 222 (dimension L1 shown in FIG. 12) of about 178 mm (about 7 inches), and screen portion 217 may have a length (dimension L2 shown in FIG. 12) of at least 127 millimeters (5 inches) inches, such as about 147 mm (about 5.77 inches). A longer length of screen portion 217 may provide the ability to use filter screen assembly 212 more times before all of the apertures 218 are clogged by sinuate debris, for example. Any suitable length may be used in various embodiments.

Adipose tissue particle processing systems 10 and 210 described herein allow adipose tissue material to be micro-fragmented (“sized”) to a controllable fat aspirate particle size, with easy connections of components. In various embodiments, the systems minimize contamination, spillage, and infection issues, while maintaining an essentially closed system during the processing of tissue and/or fluid, and/or provide a fast and convenient way to transfer adipose tissue to be filtered/sized into a vessel or container for additional sizing and/or for use in an autologous fat transfer procedure.

While various components of adipose tissue particle processing systems 10 and 210 are shown and/or described in the exemplary embodiments herein as integrated, connected, or separate components, it should be understood that in alternative embodiments, components may be integrally formed, connected, and/or separated in different ways than are shown and described herein, all within the scope and spirit of the present invention. Similarly, the sizes and dimensions of components, both in terms of absolute sizes and relative sizes with respect to other components, may be varied from what is shown and described herein, all within the scope of the present invention.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein.

What is claimed is:

1. A method of processing adipose tissue material to create and collect fat aspirate particles in a container, the fat aspirate particles having particle diameters less than or equal to a selected size, the method comprising:
(a) inserting a filter screen assembly into an interior of the container, the filter screen assembly comprising:
an interface part configured with an opening to provide access to the interior of the container through a first port in a container lid;
a closed end opposite to the interface part; and
a screen portion between the interface part and the closed end, the screen portion including a plurality of apertures having diameters of the selected size, the screen portion being supported in the interior of the container via insertion of the filter screen assembly;
(b) providing a volume of the adipose tissue material in a syringe, with a transfer cannula having its proximal end connected to the syringe;
(c) inserting the transfer cannula into the opening in the interface part of the filter screen assembly, and positioning the transfer cannula within the filter screen assembly so that a distal end of the transfer cannula is inside the screen portion of the filter screen assembly;
(d) depressing a plunger of the syringe to expel the adipose tissue material from the distal end of the transfer cannula and through the apertures of the screen portion of the filter screen assembly into the container, thereby processing the adipose tissue material into processed adipose tissue material having the fat aspirate particles with particle diameters less than or equal to the selected size;
(e) providing an additional volume of the adipose tissue material in the syringe;
(f) evaluating whether the apertures of the screen portion of the filter screen assembly are clogged with debris from the adipose tissue material, and in response to determining clogging, replacing the filter screen assembly with a replacement filter screen assembly; and
(g) repeating steps (c)-(f) until a desired amount of processed fat aspirate particles having particle diameters less than or equal to the selected size are collected in the container.

2. The method of claim 1, wherein the interface part of the filter screen assembly comprises a female luer, and inserting the transfer cannula into the opening in the interface part of the filter screen assembly comprises inserting the transfer cannula into the female luer of the interface part.

3. The method of claim 1, further comprising:
(h) extracting the processed adipose tissue material from the canister through a second port located near a bottom of the canister.

4. The method of claim 1, wherein the closed end of the filter screen assembly comprises a threaded cap that is threadedly attached to the screen portion.

5. The method of claim 1, wherein the plurality of apertures in the screen portion have diameters of 1500 microns, and the adipose tissue material is processed via the screen portion of the filter screen assembly so that the fat aspirate particles in the processed adipose tissue material have diameters of less than 1500 microns.

6. The method of claim 1, wherein the plurality of apertures in the screen portion have diameters of 1200 microns, and the adipose tissue material is processed via the screen portion of the filter screen assembly so that the fat aspirate particles in the processed adipose tissue material have diameters of less than 1200 microns.

7. The method of claim 1, wherein the screen potion of the filter screen assembly has a length of at least 127 millimeters.

8. The method of claim 1, wherein the canister has a volume of at least 1000 milliliters.

9. The method of claim 1, wherein the step of inserting the transfer cannula into the opening in the interface part of the filter screen assembly, and positioning the transfer cannula within the filter screen assembly so that a distal end of the transfer cannula is inside the screen portion of the filter screen assembly, comprises:
positioning the transfer cannula within the filter screen assembly to an extent that leaves selected apertures of the screen portion of the filter screen assembly that are unclogged by sinuate debris unobstructed by the transfer cannula.

10. An adipose tissue processing system for processing adipose tissue material to create and collect fat aspirate particles in a container, the fat aspirate particles having particle diameters less than or equal to a selected size, the system comprising:
a filter screen assembly that is insertable into an interior of the container, the filter screen assembly comprising:
an interface part configured with an opening to provide access to the interior of the container through a first port in a container lid;
a closed end opposite to the interface part; and
a screen portion between the interface part and the closed end, the screen portion including a plurality of apertures having diameters of the selected size, the screen portion being configured to be supported in the interior of the container via insertion of the filter screen assembly into the container; and
a syringe configured to be fillable with a volume of the adipose tissue material, and a transfer cannula having a proximal end connected to the syringe, the syringe and transfer cannula being configured to be inserted into the opening in the interface part of the filter screen assembly, to allow positioning of the transfer cannula within the filter screen assembly so that a distal end of the transfer cannula is inside the screen portion of the filter screen assembly, such that expelling the adipose tissue material from the syringe through the transfer cannula will cause the adipose tissue material to pass through the apertures of the screen portion of the filter screen assembly into the container, thereby processing the adipose tissue material into processed adipose tissue material having the fat aspirate particles with particle diameters less than or equal to the selected size.

11. The adipose tissue processing system of claim 10, wherein the interface part of the filter screen assembly comprises a female luer configured to receive the transfer cannula into the opening in the interface part of the filter screen.

12. The adipose tissue processing system of claim 10, wherein the canister comprises a second port located near a bottom thereof.

13. The adipose tissue processing system of claim 10, wherein the closed end of the filter screen assembly comprises a threaded cap that is threadedly attached to the screen portion.

14. The adipose tissue processing system of claim 10, wherein the plurality of apertures in the screen portion have diameters of 1500 microns.

15. The adipose tissue processing system of claim 10, wherein the plurality of apertures in the screen portion have diameters of 1200 microns.

16. The adipose tissue processing system of claim 10, wherein the screen potion of the filter screen assembly has a length of at least 127 millimeters.

17. The adipose tissue processing system of claim 10, wherein the canister has a volume of at least 1000 milliliters.
